(19) United States
(12) Patent Application Publication
(10) Pub. No.: US 2007/0298102 A1
(43) Pub. Date: Dec. 27, 2007

(54) Extended Release Pharmaceutical Composition of Celecoxib

(76) Inventor: Aleksandra Dumicic, Zagreb (HR)

Correspondence Address: BUCHANAN, INGERSOLL & ROONEY PC, POST OFFICE BOX 1404, ALEXANDRIA, VA, 22313-1404, US

(21) Appl. No.: 11/791,122

(22) PCT Filed: Nov. 23, 2005
(86) PCT No.: PCT/GB05/04500

§ 371 Date: Jul. 26, 2007

(30) Foreign Application Priority Data
Nov. 23, 2004 (GB) 0425728.3
Jul. 27, 2005 (GB) 0515315.0

(51) Int. Cl. A61K 9/30; A61K 31/415; A61P 29/00

(52) U.S. Cl. 424/465; 514/406

(57) ABSTRACT

The invention concerns new pharmaceutical agents comprising the known medicinal celecoxib for oral administration and adapted to release the celecoxib in a controlled manner over an extended period, typically for a period of up 10-12 hours or more. The invention also includes a method for the production of the new pharmaceutical agents and a method for their use to achieve the desirable therapeutic effects of celecoxib, for example in the management of pain.

### FIELD OF THE INVENTION

The invention concerns new pharmaceutical agents containing the known medicinal celecoxib, and more particularly, new extended release pharmaceutical compositions of celecoxib for oral administration adapted to release the celecoxib in a controlled manner over an extended period, typically for a period of up 10-12 hours but still provide sufficient immediate release of celecoxib to achieve a physiologically significant effect. The invention also includes a method for the production of the new pharmaceutical agents and a method for their use to achieve the desirable therapeutic effects of celecoxib, for example in the management of pain.

### BACKGROUND TO THE INVENTION

Celecoxib is the approved name for 4-[5-(4-methylphenyl)-3-(trifluoromethyl)-1H-pyrazol-1-yl]benzenesulfonamide and is described in U.S. patent Ser. No. 576,0068 (Talley et al.). It has been used in the treatment and prevention of chronic painful inflammatory diseases such as rheumatoid arthritis and osteoarthritis. Its therapeutic effects including its anti-inflammatory and analgesic properties have been attributed to its inhibition of prostaglandin synthesis primarily by selective inhibition of cyclo-oxygenase-2 (COX-2). These therapeutic effects are frequently achieved with a reduction in the adverse side-effects such as gastrointestinal disturbances which are often associated with non-selective cyclo-oxygenase inhibitors such as acetylsalicylic acid, diclofenac, naproxen, ibuprofen and similar agents.

The general preparation of pharmaceutical compositions containing celecoxib is described in several patents including PCT International publication no. WO 0032189. These compositions release the celecoxib quickly, but do not provide for its continued release over an extended period resulting in the need to adopt at least a twice daily dosing regimen when controlling pain in patients. PCT International publication no. WO 01/45705 describes oral compositions of celecoxib with a release-extending polymer which are said to permit once-a-day administration in treating COX-2 mediated medical conditions and disorders. Further, US patent application publication no. US 2004/0242640 describes complex formulations of celecoxib with components containing celecoxib of specific particle sizes.

Notwithstanding, previous attempts to solve this problem, there is a continuing need for a simple oral pharmaceutical composition capable of immediately releasing sufficient celecoxib to achieve its desirable therapeutic effects (such as analgesic effects) within a short time, and continuing the release of further celecoxib over an extended period, thereby providing both rapid and continuing control of pain. It has now-been unexpectedly discovered that this problem may be solved using a novel, coated pharmaceutical composition in which both the core and the coating of the composition contain some celecoxib.

### SUMMARY OF THE INVENTION

According to the invention there is provided a novel extended release pharmaceutical composition of celecoxib which comprises a largely water-insoluble core and a pharmaceutically acceptable hydrophilic coating, wherein the core comprises celecoxib and one or more pharmaceutically acceptable excipients, and the coating comprises celecoxib and one or more pharmaceutically acceptable solubilisers.

The compositions of the invention provide a combination of both immediate and extended release of celecoxib leading to rapid onset and sustained provision of therapeutic effects. They provide long-acting oral formulations which can be administered once daily resulting in benefits including improved therapeutic efficacy (for example in controlling pain), reduction in potential adverse effects and increased patient compliance.

### BRIEF DESCRIPTION OF DRAWINGS

FIG. 1 shows typical release profiles for celecoxib from illustrative compositions of the invention described in Examples 1-3 (referred to as AC1, AC2 and AC3 respectively) compared to the limits specified for the compositions described in PCT International publication no. WO 01/45705, using UV spectroscopy as the analytical probe in phosphate buffer as described in Example 4 hereof

FIG. 2 shows an X-ray powder diffraction (XRPD) pattern for a representative sample of celecoxib Form N which Form is preferably used as the active ingredient in the compositions of the invention. The XRPD was measured using CuKα radiation on a powder sample collected using a PANalytical X'PertPRO powder diffractometer.

FIG. 3 shows the release of celecoxib determined by UV spectroscopy at λ=255 nm on a sample in phosphate buffer as described in Example 6 hereof.

### DETAILED DESCRIPTION

According to the invention, typical pharmaceutically acceptable excipients which may be present in the core include, for example, ethylcellulose and similar water-insoluble cellulose derivatives, and polymethacrylates, such as the Eudragit™ NE, Eudragit™ RS and Eudragit™ RL types which are commercially available from Rohm Polymers, Darmstadt, Germany, and long-chain fatty acid derivatives such as magnesium stearate, sodium stearyl fumarate, glyceryl tristearate, glyceryl behenate and glyceryl palmitostearate.

One or more additional pharmaceutical excipients such as diluents, carriers, lubricants or compression aids, for example, lactose, microcrystalline cellulose, colloidal silicon dioxide, talc, hydrogenated vegetable oil (for example that commercially available as Lubritab™) or magnesium stearate or the like, may be present in the core or another part of the overall compositions of the invention.

Typical pharmaceutically acceptable solubilisers in the coating include, for example, anionic surfactants such as sodium lauryl sulphate. Other constituents which may be present in the coating include, for example, hypromellose and polysorbatum, but other well know coating constituents may also be included. In particular, the coating may also contain one or more conventional pharmaceutically acceptable pigments such as titanium dioxide or iron oxide and a sweetener or taste masking agent may also optionally be present.

The compositions of the invention may be in single unit dosage form such as tablets or capsules, of which tablets are preferred. Alternatively they may be in multiple unit dosage form, in the form of granules, pellets or mini-tablets.

The term “pharmaceutically acceptable” refers to a constituent of a pharmaceutical composition that is generally known to be suitable for pharmaceutical use and safe for administration to humans and animals.

Without wishing to be bound by any theory, it is believed that the largely water-insoluble core forms a matrix that controls diffusion of water into the composition. This water dissolves the celecoxib within a certain time enabling release of dissolved material in a sustained manner. For preparing the core, the celecoxib active ingredient may either be granulated with the water-insoluble material or else homogenised into an intimate mixture that can be further used in the manufacture of the oral dosage forms.

Inclusion of celecoxib and a solubiliser such as an anionic surfactant like sodium lauryl sulphate into the coating is believed to result in the initial fast release of the celecoxib to achieve the required biological effects within a short time.

The compositions according to the invention may be manufactured by standard pharmaceutical processes well known in the art, for example as illustrated in the accompanying Examples. It will be understood that the order of adding particular constituents and excipients in the manufacture of compositions of the invention may be varied according to standard practice. However, in general it is preferred to mix the celecoxib active ingredient with one or more of the lipid pharmaceutically acceptable ingredients, for example, with the Eudragit™ polymer when present.

The particular amount of celecoxib in the core and in the coating of a composition according to the invention may typically be in the general range 5-75% by weight. However, preferred compositions include those in which the core contains, for example about 70-85%, and the coating about 15-30%, by weight of celecoxib active ingredient. Especially preferred compositions include those in which the core contains, for example, about 80% and the coating about 20% by weight of celecoxib active ingredient. The overall amount of celecoxib per unit pharmaceutical composition (for example per tablet) is about 100-700 mg, and preferably about 100-300 mg.

Compositions according to the invention provide at least 20% (and preferably 30% release) of celecoxib within 1 hour with extended release of celecoxib over a further 15 hours, making compositions of the invention suitable for single daily dosing. The release of celecoxib may be measured using standard techniques well known in the pharmaceutical arts such as those provided for checking dissolution of active ingredients in Pharmacopoeias such as the US Pharmacopoeia.

The invention also provides a novel extended release pharmaceutical composition comprising celecoxib in the amount of 50-700 mg per unit dosage form (and preferably 50-250 mg per unit dosage form), dispersed between a largely water-insoluble core and a hydrophilic coating.

It may also be desirable for the core to include a further component which comprises a largely water insoluble, largely water impermeable substance, such as magnesium stearate, sodium stearyl fumarate, glyceryl tristearate, glyceryl behenate or glyceryl palmitostearate, itself mixed with celecoxib and such a pharmaceutical composition is provided as a further feature of the invention. Typically in such a composition the celecoxib in the further component of the core constitutes about 10-30% of the overall weight of celecoxib in the composition.

The invention also provides a process for the manufacture of a new extended release pharmaceutical composition of celecoxib as defined above which comprises the steps of preparing the core by mixing celecoxib with one or more additional pharmaceutically acceptable excipients and then applying the hydrophilic coating in a conventional manner.

The invention also provides a method for delivering a COX-2 inhibitory amount of celecoxib over an extended period to an animal requiring such treatment for example in the management of pain which comprises administering to such animal an effective amount of a novel extended release composition of the invention as defined above.

The celecoxib used as active ingredient may be in any convenient physical form which remains stable during and following formulation. The celecoxib is generally used as a powder with particle size with d₉₀ of about 50-200 μm and preferably of about 50-80 μm.

A particularly suitable form of celecoxib for use in the compositions of the invention is that referred to as celecoxib Form N. This Form N is essentially free of amorphous and other polymorphic forms and has characteristic X-ray diffraction pattern peaks, expressed in d values, at about 16.0 Å, 15.3 Å, 12.3 Å, 10.6 Å, 8.0 Å, 6.5 Å, and 5.4 Å. Celecoxib Form N may be obtained by heating a stirred suspension of the known, thermodynamically most stable celecoxib Form III in n decane or n-tetradecane at about 165° C. to give an emulsion. This is then cooled with stirring to about 145° C., reheated to about 165° C. and then cooled to about 110° C. The crystalline solid obtained is separated from the resultant suspension and dried under vacuum at 100° C. to give celecoxib Form N.

The invention provides as a still further and preferred feature an extended release coated pharmaceutical composition as defined above which is characterised in that the celecoxib active ingredient is celecoxib Form N.

The invention will now be illustrated by the following non-limiting Examples:

### EXAMPLES

### Example 1

| Component | Core | Coating |
|---|---|---|
| Celecoxib (%) | 80 | 20 |

| Composition of the tablets | (mg) |
|---|---|
| Celecoxib* | 100.00 |
| Microcrystalline cellulose | 169.20 |
| Lactose | 160.00 |
| Eudragit ™ RS | 20.00 |
| Glyceryl tristearate | 11.00 |
| Lubritab ™ | 11.00 |
| Calcium hydrogen phosphate | 10.00 |
| Colloidal silicon dioxide | 3.80 |
| Magnesium stearate | 5.00 |
| Talc | 2.08 |
| Hypromellose | 3.30 |
| Polysorbatum 80 | 1.50 |
| Sodium lauryl sulphate | 0.08 |
| Titanium dioxide | 2.90 |
| Iron oxide, red | 0.08 |
| Iron oxide, yellow | 0.06 |

*d₉₀ = 62 μm, d₅₀ = 17 μm (mixture of various batches)

Preparation of Core

Celecoxib was intermixed with lactose and parts of microcrystalline cellulose and colloidal silicon dioxide and granulated with an aqueous dispersion of Eudragit™ RS. Wet granules were dried in a fluid-bed dryer, and then milled through a 20 mesh (0.8 mm) screen to obtain appropriate size distribution of the granules suitable for compression.

Glyceryl tristearate, Lubritab™, calcium hydrogen phosphate and the remaining parts of microcrystalline cellulose and colloidal silicon dioxide, were blended 20 min prior to sieving through a 30 mesh (0.6 mm) sieve.

Magnesium stearate, screened through a 30 mesh (0.6 mm) sieve was added to the core component above. The final blend was homogenised for another 5 minutes and then compressed into tablets.

Coating

The remaining part of celecoxib was dispersed in an aqueous suspension of talc, hypromellose, polysorbatum, sodium lauryl sulphate, titanium dioxide and iron oxides, and used for tablet coating.

### Example 2

| Components | Core | Coating |
|---|---|---|
| Celecoxib (%) | 80 | 20 |

| Composition of the tablets | (mg) |
|---|---|
| Celecoxib (d₉₀ = 68 μm, d50 = 13 μm) | 100.00 |
| Microcrystalline cellulose | 169.20 |
| Lactose | 160.00 |
| Eudragit ™ RS | 20.00 |
| Glyceryl tristearate | 5.00 |
| Lubritab ™ | 5.00 |
| Calcium hydrogen phosphate | 10.00 |
| Colloidal silicon dioxide | 3.80 |
| Magnesium stearate | 5.00 |
| Talc | 2.08 |
| Hypromellose | 3.30 |
| Polysorbatum 80 | 1.50 |
| Sodium lauryl sulphate | 0.08 |
| Titanium dioxide | 2.90 |
| Iron oxide, red | 0.08 |
| Iron oxide, yellow | 0.06 |

Preparation of Core

Celecoxib was intermixed with lactose and parts of microcrystalline cellulose and colloidal silicon dioxide and granulated with an aqueous dispersion of Eudragit™ RS. Wet granules were dried in a fluid-bed dryer, and then milled through a 20 mesh (0.8 mm) screen to obtain appropriate size distribution of the granules suitable for compression. Glyceryl tristearate, Lubritab™, calcium hydrogenphosphate and the remaining parts of microcrystalline cellulose and colloidal silicon dioxide, were blended for 20 min prior to sieving through a 30 mesh (0.6 mm) sieve.

Magnesium stearate, screened through a 30 mesh (0.6 mm) sieve was added to the core component above. The final blend was homogenised for another 5 minutes and then compressed into tablets.

Coating

The remaining part of celecoxib was dispersed in an aqueous suspension of talc, hypromellose, polysorbatum, sodium lauryl sulphate, titanium dioxide and iron oxides, and used for tablet coating.

### Example 3

| Components | Core | Coating |
|---|---|---|
| Celecoxib (%) | 80 | 20 |

| Composition of the tablets | (mg) |
|---|---|
| Celecoxib (d₉₀ = 68 μm, d50 = 13 μm) | 100.00 |
| Microcrystalline cellulose | 169.20 |
| Lactose | 160.00 |
| Eudragit ™ RS | 20.00 |
| Glyceryl tristearate | 1.00 |
| Lubritab ™ | 1.00 |
| Calcium hydrogen phosphate | 10.00 |
| Colloidal silicon dioxide | 3.80 |
| Magnesium stearate | 5.00 |
| Talc | 2.08 |
| Hypromellose | 3.30 |
| Polysorbatum 80 | 1.50 |
| Sodium lauryl sulphate | 0.08 |
| Titanium dioxide | 2.90 |
| Iron oxide, red | 0.08 |
| Iron oxide, yellow | 0.06 |

Preparation of Core

Celecoxib was intermixed with lactose and part of the microcrystalline cellulose and colloidal silicon dioxide and granulated with an aqueous dispersion of Eudragit™ RS. Wet granules were dried in a fluid-bed dryer, and then milled through a 20 mesh (0.8 mm) screen to obtain appropriate size distribution of the granules suitable for compression. Glyceryl tristearate, Lubritab™, calcium hydrogen phosphate and the remaining parts of the microcrystalline cellulose and colloidal silicon dioxide were blended 20 min prior to sieving through a 30 mesh (0.6 mm) sieve. Magnesium stearate, screened through a 30 mesh (0.6 mm) sieve was added to the core component. The final blend was homogenised for another 5 minutes and then compressed into tablets.

Coating

The remaining part of celecoxib was dispersed in an aqueous suspension of talc, hypromellose, polysorbatum, sodium lauryl sulphate, titanium dioxide and iron oxides, and used for tablet coating.

### Example 4

[This example describes release studies on the illustrative coated mono-component formulations of the invention described in Examples 1-3 above.]

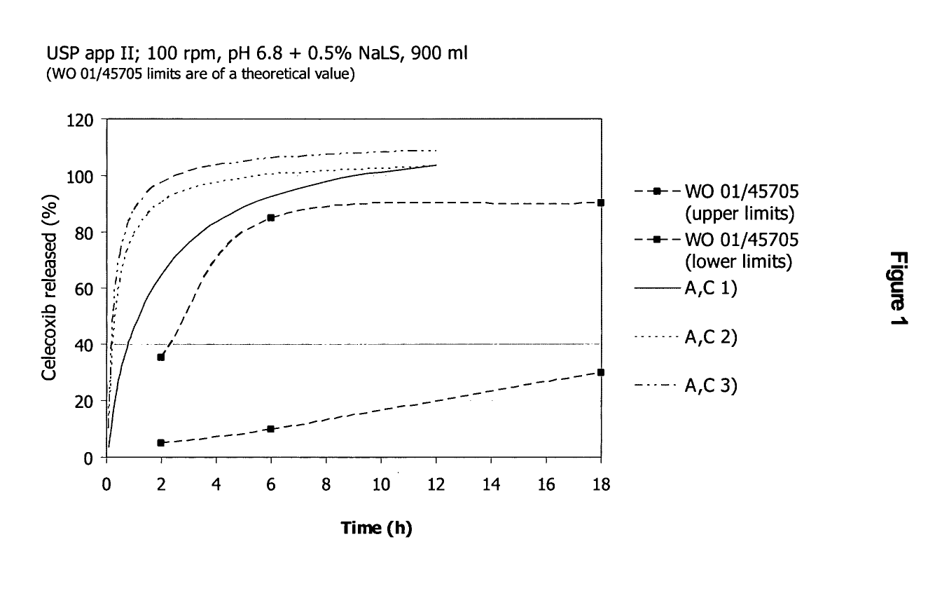

Samples of formulation were placed in 900 ml of 37° C. phosphate buffer pH 6.8 containing 0.5% sodium lauryl sulphate (“NaLS”) using the procedure described for method II in the US Pharmacopoeia (100 rpm). The release of celecoxib was determined by UV spectroscopy (λ=255 nm). The results obtained are shown in Table 1 below and in FIG. 1.

TABLE 1
celecoxib release in phosphate buffer

| Time | Ex 1 | Ex 2 | Ex 3 |
|---|---|---|---|
| 0.083 | 3.6 | 10.5 | 10.3 |
| 0.25 | 16.6 | 42.4 | 51.9 |
| 0.5 | 29.8 | 63.0 | 72.3 |
| 0.75 | 38.9 | 73.3 | 82 |
| 1 | 46.2 | 79.5 | 87.8 |
| 1.5 | 57.0 | 86.5 | 94.1 |
| 2 | 65.0 | 90.6 | 97.7 |
| 2.5 | 71.4 | 93.3 | 100.1 |
| 3 | 76.3 | 95.2 | 101.8 |
| 3.5 | 80.5 | 96.6 | 103 |
| 4 | 83.8 | 97.6 | 103.8 |
| 5 | 89.0 | 99.3 | 105.1 |
| 6 | 92.7 | 100.3 | 106 |
| 7 | 95.4 | 100.9 | 106.6 |
| 8 | 97.8 | 101.6 | 107.2 |
| 9 | 99.8 | 102.0 | 107.6 |
| 10 | 101.1 | 102.4 | 108 |
| 11 | 102.5 | 102.8 | 108.4 |
| 12 | 103.8 | 103.1 | 108.7 |

### Example 5

This Example describes the preparation of an alternative composition of the invention: in which the core contains an additional water insoluble, water impermeable substance component which has itself been blended with celecoxib.

| Composition of the tablets | (mg) |
|---|---|
| Celecoxib | 100.00 |
| Microcrystalline cellulose | 200.00 |
| Lactose | 200.00 |
| Eudragit ™ RS | 40.00 |
| Glyceryl tristearate | 60.00 |
| Talc | 5.00 |
| Magnesium stearate | 3.00 |
| Hypromellose | 10.00 |
| Polysorbatum 80 | 1.90 |
| Sodium lauryl sulphate | 0.10 |

Preparation of Component A

Celecoxib (70 parts by weight) was intermixed with microcrystalline cellulose and lactose and granulated with an aqueous dispersion of Eudragit™ RS. Wet granules were dried in a fluid-bed dryer, and then milled through a 20 mesh (0.8 mm) screen to obtain appropriate size distribution of the granules suitable for compression.

Preparation of Component B

Celecoxib (10 parts by weight) was mixed with glyceryl tristearate, blended 15 minutes and sieved through a 30 mesh (0.6 mm) sieve.

Compression

Components A and B above and talc were homogenised for 15 minutes. Magnesium stearate, screened through a 30 mesh (0.6 mm) sieve was added to the final blend and homogenised for another 5 minutes. The final blend was compressed into tablets.

Hydrophilic Coating

The remaining celecoxib (20 parts by weight) was dispersed in an aqueous suspension of hypromellose, polysorbatum and sodium lauryl sulphate and used for tablet coating. If desired, various pigments, such as titanium dioxide or iron oxides, may be added to the hydrophilic coating.

Note: the celecoxib used in Examples 1-3 and 5 may conveniently be celecoxib Form N which may be prepared as follows:

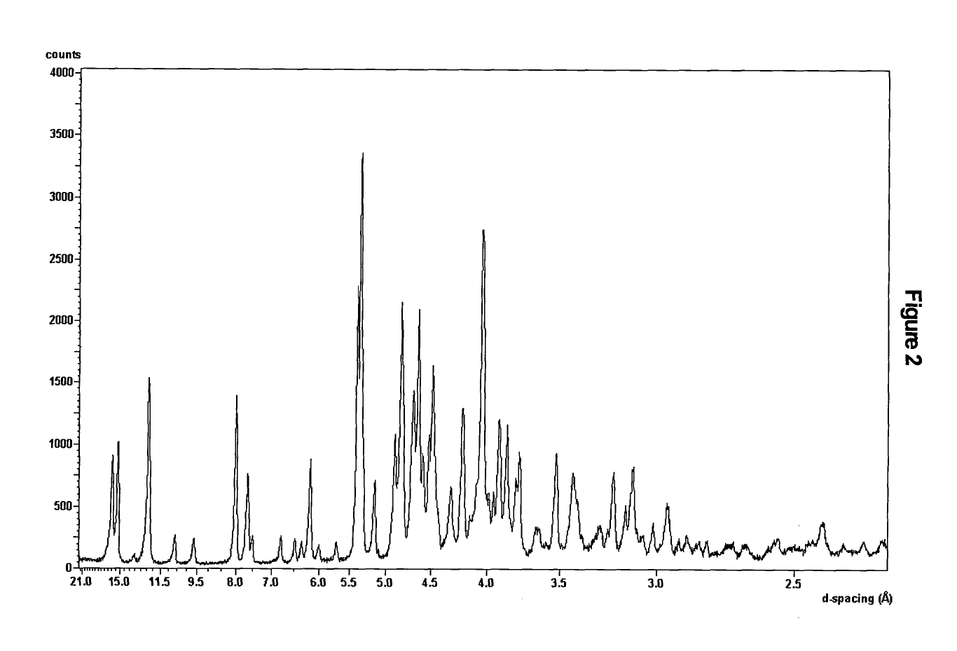

Celecoxib Form III (2.5 g) is suspended in 50 ml of n-tetradecane and then heated to about 165° C. while stirring. The emulsion obtained is stirred at the same temperature for about 15 min and then cooled to about 145° C. It is then reheated to about 165° C. and then cooled to about 110° C. The resultant suspension is separated by filtration and the crystals obtained are dried at 100° C. under the vacuo for 12 hours to yield celecoxib Form N. FIG. 2 shows an X-ray powder diffraction (XRPD) pattern for a representative sample of celecoxib Form N measured using CuKα radiation on a powder sample collected using a PANalytical X'PertPRO powder diffractometer. The pattern has characteristic peak position (expressed in d values) at 16.0±0.2 Å, 15.3±0.2 Å, 12.3±0.2 Å and 10.6±0.2 Å, and further characteristic peaks at 8.0±0.2 Å, 6.5±0.1 Å, and 5.4±0.1 Å.

The starting celecoxib Form III may itself be produced, for example, as described in US patent application publication no. 2004/0087640A starting from celecoxib produced by any known process, for example that described in example 1 of U.S. Pat. No. 5,910,597.

### Example 6

[This Example describes release studies on a typical composition of the invention made according to Example 5.]

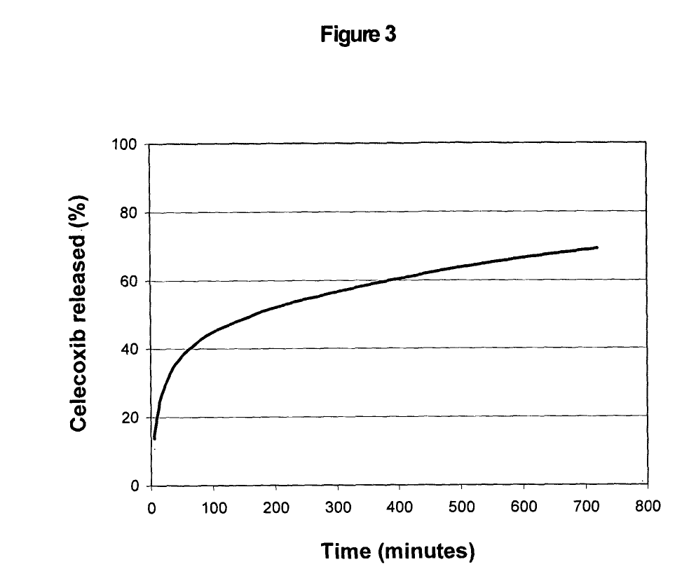

A sample of the tablets from Example 5 was placed in 900 ml of 37° C. phosphate buffer pH6.8 containing 0.5% sodium lauryl sulphate using the procedure described for method I in the US Pharmacopoeia. The release of celecoxib was determined by UV spectroscopy at λ=255 nm as shown in the attached FIG. 3. The results show the immediate release of >20% of celecoxib in the first 15 minutes followed by extended release over the next 10 hours with >30% of celecoxib remaining.

1. An extended release pharmaceutical composition of celecoxib which comprises a largely water-insoluble core and a pharmaceutically acceptable hydrophilic coating, wherein the core comprises celecoxib and one or more pharmaceutically acceptable excipients and the coating comprises celecoxib and one or more pharmaceutically acceptable solubilisers or coating agents.

2. A composition according to claim 1 in which the solubiliser is an anionic surfactant such as sodium lauryl sulphate.

3. A composition according to claim 1 in which the coating comprises one or more coating agents.

4. A composition according to claim 1 in which the pharmaceutically acceptable excipients which may be present are selected from one or more ethylcellulose and similar water-insoluble cellulose derivatives, polymethacrylate derivates of the Eudragit™ type, and long-chain fatty acid derivatives.

5. A composition according to claim 4 which comprises a Eudragit™ RS type polymethacrylate.

6. A composition according to claim 4 wherein the long chain fatty acid derivative is selected from one or more of magnesium stearate, sodium stearyl fumarate, glyceryl tristearate, glyceryl behenate and glyceryl palmitostearate.

7. A composition according to claim 1 which comprises one or more additional pharmaceutical excipients selected from diluents, carriers, lubricants and compression aids.

8. A composition according to claim 1 in which the celecoxib is celecoxib Form N.

9. A composition according to claim 1 wherein the core includes a further component which comprises a largely water insoluble, largely water impermeable substance itself mixed with celecoxib.

10. A process for the manufacture of an extended release pharmaceutical composition of celecoxib as claimed in claim 1 comprising the steps of preparing the core by mixing celecoxib with one or more additional pharmaceutically acceptable excipients, and then applying the hydrophilic coating.

11. A composition according to claim 1 in which the coating comprises one or more coating agents.

12. A composition as claimed in claim 3 wherein the coating agent is selected from the group consisting of hypromellose, polysobatum, pigments, sweeteners, taste masking agents, and mixtures of the foregoing.

13. A composition according to claim 2 in which the pharmaceutically acceptable excipients which may be present are selected from one or more ethylcellulose and similar water-insoluble cellulose derivatives, polymethacrylate derivates of the Eudragit™ type, and long-chain fatty acid derivatives.

14. A composition according to claim 3 in which the pharmaceutically acceptable excipients which may be present are selected from one or more ethylcellulose and similar water-insoluble cellulose derivatives, polymethacrylate derivates of the Eudragit™ type, and long-chain fatty acid derivatives.

15. A composition according to claim 11 in which the pharmaceutically acceptable excipients which may be present are selected from one or more ethylcellulose and similar water-insoluble cellulose derivatives, polymethacrylate derivates of the Eudragit™ type, and long-chain fatty acid derivatives.

16. A composition according to claim 12 in which the pharmaceutically acceptable excipients which may be present are selected from one or more ethylcellulose and similar water-insoluble cellulose derivatives, polymethacrylate derivates of the Eudragit™ type, and long-chain fatty acid derivatives.

17. A composition according to claim 5 wherein the long chain fatty acid derivative is selected from one or more of magnesium stearate, sodium stearyl fumarate, glyceryl tristearate, glyceryl behenate and glyceryl palmitostearate.

18. A composition according to claim 2 which comprises one or more additional pharmeceutical excipients selected from diluents, carriers, lubricants and compression aids.

19. A composition according to claim 2 in which the celecoxib is celecoxib Form N.

20. A composition according to claim 2 wherein the core includes a further component which comprises a largely water insoluble, largely water impermeable substance itself mixed with celecoxib.
